(19) United States
(12) Patent Application Publication
(10) Pub. No.: US 2021/0295184 A1
(43) Pub. Date: Sep. 23, 2021

(54) Predicting And Mitigating Athlete Injury Risk

(71) Applicant: InPlay Ltd., Ramat Gan (IL)

(72) Inventors: Eyal Eliakim, Givatayim (IL); Tal Brown, Palo Alto, CA (US)

(21) Appl. No.: 17/206,912

(22) Filed: Mar. 19, 2021

Related U.S. Application Data
(60) Provisional application No. 62/992,683, filed on Mar. 20, 2020.

(51) Int. Cl. G06N 5/04; A63B 71/06; A63B 24/00

(52) U.S. Cl. CPC G06N 5/04; A63B 2024/0065; A63B 24/0062; A63B 71/0616

(57) ABSTRACT

The present invention extends to methods, systems, and computer program products for predicting and mitigating athlete injury risk. In general, performance parameter data for athletes can be captured and maintained over time. The performance parameter data can be utilized in at least two different ways. In one aspect, performance parameter data is utilized to detect athletes at increased risk for injury and/or indicate why athletes are at increased risk for injury. In another aspect, performance parameter data is utilized to recommend activities (e.g., training sessions) for athletes that are injured or at increased risk for injury. The recommended activities can reduce athlete injury risk while also minimizing reduction in athlete workload. Recommended activities can be tailored based on injury type, injury severity, risk type, risk level, competition schedule, etc.

### RELATED APPLICATIONS

This application claims the benefit of U.S. Provisional Application Ser. No. 62/992,683 entitled “Detecting And Mitigating Athlete Injury Risk” and filed Mar. 20, 2020, which is hereby incorporated herein by reference in its entirety

### TECHNICAL FIELD

The present disclosure relates generally to predicting and mitigating athlete injury risk. Activities (e.g., training sessions) that both sufficiently reduce athlete injury risk and optimize athlete workload, for example, based on a physical condition of the athlete, can be recommended.

### BACKGROUND

Athletes, especially those competing professionally, can be subject to various physical conditions, including injuries, during training and/or in matches/games. Depending on severity, injuries may or may not result in an athlete having to sit out of training and/or matches/games. Other physical conditions may not rise to the level of an injury but may be an indication that an athlete is at higher risk for injury.

Injuries as well as other physical conditions can decrease an athlete's performance in training and/or in matches/games. In some cases, a performance decrease is readily apparent/detectable to coaches, trainers, etc. For example, an athlete is limping, is unable to run, has difficulty changing body position, etc. In other cases, a performance decrease (or cause thereof) is not readily apparent/detectable to coaches, trainers, etc. For example, an athlete may have pain in his leg muscle but can still accelerate and/or run as normal.

When injuries are other physical conditions are detected, a typical response may be to have the athlete sit out of training and/or rest in preparation for the next match/game. Further, undetected physical conditions can lead to injury during subsequent training or matches/games.

### BRIEF DESCRIPTION OF THE DRAWINGS

The specific features, aspects and advantages of the present invention will become better understood with regard to the following description and accompanying drawings where:

FIG. 1 illustrates an example block diagram of a computing device.

FIG. 2 illustrates an example computer architecture of predicting and mitigating athlete injury risk.

FIGS. 3A and 3B illustrates a flow chart of an example method for predicting and mitigating athlete injury risk.

FIGS. 4A and 4B illustrate an example computer architecture for collecting data and making injury risk predictions.

FIGS. 5A and 5B illustrate another example computer architecture for collecting data and making injury risk predictions.

### DETAILED DESCRIPTION

The present invention extends to methods, systems, and computer program products for predicting and mitigating athlete injury risk. Activities (e.g., training sessions) can be recommended for an athlete to both sufficiently reduce athlete injury risk and optimize athlete workload, for example, based on the athlete's physical performance.

Aspects of the invention can be implemented to assist athletes, coaches, trainers, etc. participating in and/or associated with any of a variety of sports, including soccer, basketball, baseball, softball, football, lacrosse, volley-ball, rugby, track and field, cross country, other races, tennis, wrestling, skiing, boxing, cycling, etc.

In general, performance parameter data (e.g., workload, injury history, schedules, training plans, etc.) for athletes can be captured and maintained over time. The performance parameter data can be utilized in at least two different ways. In one aspect, performance parameter data is utilized to detect athletes at increased risk for injury and/or indicate why athletes are at increased risk for injury. In another aspect, performance parameter data is utilized to recommend activities (e.g., training sessions) for athletes that are injured or at increased risk for injury. The recommended activities can reduce athlete injury risk while also minimizing reduction in athlete workload. Recommended activities can be tailored based on injury type, injury severity, risk type, risk level, competition schedule, etc.

Often when an athlete is injured or at increased risk for injury, the recommended course is simply to have the athlete rest, sit out of activities, or “underload” the athlete. However, depending on circumstances, resting, sitting out, or underloading may not be optimal or even appropriate. For example, it may be that an athlete is at risk for an injury but still able to play in games/matches. If the next match/game is a number of days away, resting the athlete may reduce the athlete's conditioning for the next match/game. Reduced conditioning can increase injury risk.

As such, it may be beneficial for the athlete to perform activities that both optimize performance (e.g., maintain conditioning) and also mitigate risk of injury. Through reference to athlete performance parameter data, training sessions can be tailored to simultaneously optimize workload and minimize injury risk. For example, drills can be isolated and varied in intensity.

Risk of injury can be quantified by considering athlete data over different time frames, such as, for example, six months back, three months back, one month back, one week back, etc. Algorithms used to calculate risk can weight data from different time frames differently. In one aspect, more recent data can be weighted more heavily than less recent data. Athlete data can be related to movement activities (lateral movement, acceleration, running, etc.) as well as performing sport specific activities (e.g., kicking, dribbling, throwing, catching, pitching, hitting, shooting, blocking, etc.).

In one aspect, machine or deep learning classifiers are trained to detect injury risk. A classifier can be trained based on athlete physical activity, prior injuries, competition schedule, etc. Thus, for example, using pattern recognition methods, an injury in one athlete can be used to detect increased risk of the same injury in another athlete. In one aspect, a match-to-match micro-cycle phase is used to collectively represent days from last match/game and days to next match/game, such as, for example, +3/−4 (3 days from the last match/game and 4 days to the next match/game). Due at least in part to the repetitive nature of workload in a given phase of the micro-cycle, workload in a given phase can be used to predict the extent of the workload to be prescribed on the next day.

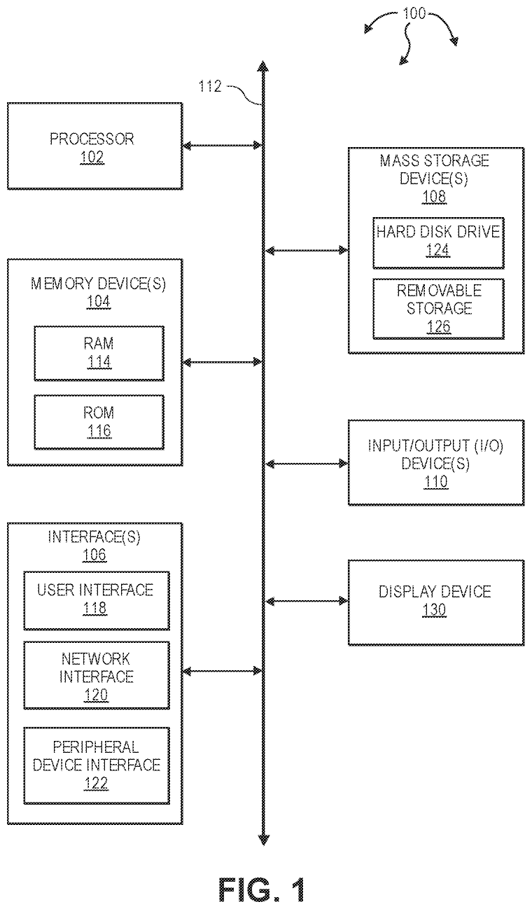

Turning to FIG. 1, FIG. 1 illustrates an example block diagram of a computing device 100. Computing device 100 can be used to perform various procedures, such as those discussed herein. Computing device 100 can function as a server, a client, or any other computing entity. Computing device 100 can perform various communication and data transfer functions as described herein and can execute one or more application programs, such as the application programs described herein. Computing device 100 can be any of a wide variety of computing devices, such as a mobile telephone or other mobile device, a desktop computer, a notebook computer, a server computer, a handheld computer, tablet computer and the like.

Computing device 100 includes one or more processor(s) 102, one or more memory device(s) 104, one or more interface(s) 106, one or more mass storage device(s) 108, one or more Input/Output (I/O) device(s) 110, and a display device 130 all of which are coupled to a bus 112. Processor(s) 102 include one or more processors or controllers that execute instructions stored in memory device(s) 104 and/or mass storage device(s) 108. Processor(s) 102 may also include various types of computer storage media, such as cache memory. Processor(s) 102 can be real or virtual and can be allocated from on-premise, cloud computing or any cloud provider resources.

Memory device(s) 104 include various computer storage media, such as volatile memory (e.g., random access memory (RAM) 114) and/or nonvolatile memory (e.g., read-only memory (ROM) 116). Memory device(s) 104 may also include rewritable ROM, such as Flash memory. Memory device(s) 104 can be real or virtual and can be allocated from on-premise, cloud computing or any cloud provider resources.

Mass storage device(s) 108 include various computer storage media, such as magnetic tapes, magnetic disks, optical disks, solid state memory/drives (e.g., Flash memory), and so forth. As depicted in FIG. 1, a particular mass storage device is a hard disk drive 124. Various drives may also be included in mass storage device(s) 108 to enable reading from and/or writing to the various computer readable media. Mass storage device(s) 108 include removable media 126 and/or non-removable media. Mass storage device(s) 108 can be real or virtual and can be allocated from on-premise, cloud computing or any cloud provider resources.

I/O device(s) 110 include various devices that allow data and/or other information to be input to or retrieved from computing device 100. Example I/O device(s) 110 include cursor control devices, keyboards, keypads, barcode scanners, microphones, monitors or other display devices, speakers, printers, network interface cards, modems, cameras, and the like. I/O device(s) 110 can be real or virtual and can be allocated from on-premise, cloud computing, or any cloud provider resources.

Display device 130 includes any type of device capable of displaying information to one or more users of computing device 100. Examples of display device 130 include a monitor, display terminal, video projection device, and the like. Display device 130 can be real or virtual and can be allocated from on-premise, cloud computing or any cloud provider resources.

Interface(s) 106 include various interfaces that allow computing device 100 to interact with other systems, devices, or computing environments as well as humans. Example interface(s) 106 can include any number of different network interfaces 120, such as interfaces to personal area networks (PANs), local area networks (LANs), wide area networks (WANs), wireless networks (e.g., near field communication (NFC), Bluetooth, Wi-Fi, etc., networks), and the Internet. Network interface 120 can connect computing device 100 to other devices and systems, including devices and systems configured to capture, store, transfer, and process athlete parameter data. Other interfaces include user interface 118 and peripheral device interface 122. Interface(s) 106 can be real or virtual and can be allocated from on-premise, cloud computing or any cloud provider. Peripheral device interface 122 can connect computing device 100 to other devices and systems, including devices and systems configured to capture, store, transfer, and process athlete parameter data.

Bus 112 allows processor(s) 102, memory device(s) 104, interface(s) 106, mass storage device(s) 108, and I/O device(s) 110 to communicate with one another, as well as other devices or components coupled to bus 112. Bus 112 represents one or more of several types of bus structures, such as a system bus, PCI bus, IEEE 1394 bus, USB bus, and so forth. Bus 112 can be real or virtual and can be allocated from on-premise, cloud computing or any cloud provider. Any of a variety of protocols can be implemented over bus 112, including protocols used to capture, store, transfer, and process athlete parameter data.

In this description and the following claims, “athlete” and “player” are used interchangeably and defined as one (a human being) that participates in and/or is preparing for participation in individual or team (e.g., competitive) sporting activities, such as, for example, track and field events, cross country, other races, soccer, tennis, basketball, baseball, softball, football, rugby, wrestling, skiing, lacrosse, boxing, volley-ball, cycling, etc. at an amateur level or professional level.

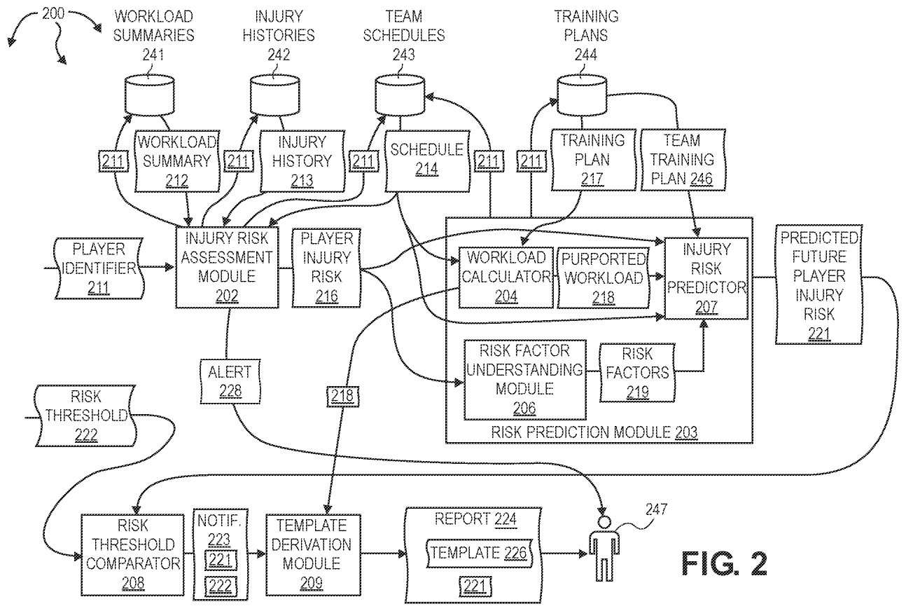

FIG. 2 illustrates an example computer architecture 200 that facilitates predicting and mitigating athlete injury risk. As depicted, computer architecture 200 includes injury risk assessment module 202, risk prediction module 203, risk threshold comparator 208, template derivation module 209, workload summaries 241, injury histories 242, team schedules 243, and training plans 244.

Workload summaries 241 (e.g., a database, file, etc.) contains individual summaries of workload data associated with prior workloads for one or more players. Prior workloads can include archived training data and/or archived match/game data. Team personnel can enter workload related data into training workload summaries 241 as workload related data is accumulated per player, for example, at the completion of training sessions and/or after games/matches. In other aspects, workload data is accessed from other systems (then possibly normalized) and summarized/stored into workload summaries 241.

Player workloads can include training sessions, other non-game/match workout activities, match/game participation, etc. performed by a player. A player workload summary can indicate what (particular drills/activities, game/match playing time, position played, etc.), when (date/time), where (physical location, in practice, in a game/match, etc.), why (workload reason), and how (group, solo, match/game simulation, intensity level, match/game, etc.) the player performed workload activities. Workload summaries can also indicate field/weather conditions, for example, wet, snow, low temperatures, high temperatures, wind, etc., associated with performed workload activities.

In one aspect, player workload summaries are stored in workload summaries 241 per player by player identifier.

Injury histories 242 (e.g., a database, file, etc.) contains individual injury histories for one or more players. Team personnel can enter player injuries into injury histories 242 as player injuries occur. In other aspects, injuries are accessed from other systems (then possibly normalized) and summarized/stored into injury histories 242. Injuries can occur during training activities, during match/game activities, or even in regular day to day activities (e.g., accidents).

A player injury history can include entries for any player injuries per player (some players may have no injuries). Each injury entry can indicate injury type (e.g., leg, arm, shoulder, back, etc.), injury severity (numerical in a range, low/mid/high, etc.), resulting performance degradations (if any), required recovery time, injury circumstances, etc. associated with a player injury. Performance degradations can indicate degradation type (e.g., reduced speed or acceleration (slower), reduced lateral mobility, decreased leg/arm strength, etc.), duration (e.g., how long did performance degradation last, how long before improvement was measured, etc.), etc. Injury circumstances can include where (e.g., physical location, in practice, in a game/match, etc.), when (date/time), how/why (e.g., lost footing, collided with other player/official, collided goal post, etc.), field/weather conditions (e.g., wet, snow, low temperatures, high temperatures, wind, etc.), what position was being played at the time of the injury, etc. associated with a player injury.

In one aspect, player injury histories are stored in injury histories 242 per player by player identifier.

Team schedules 243 (e.g., a database, file, etc.) contains a match/game schedule for one or more teams. Team personnel can enter a team schedule into team schedules 243. In other aspects, a team schedule is accessed from other systems (then possibly normalized) and summarized/stored into team schedules 243. Per team, team schedules 243 can include past, present, and future matches/games, for example, in a current or prior season. In another aspect, team schedules include events for athletes participating in individual events (e.g., races, tennis matches, etc.).

In one aspect, team schedules are stored in team schedules 243 per team. A team schedule can map/link to players that are on the team per player by player identifier.

In general, injury risk assessment module 202 is configured to assess injury risk for a player from historical player data (parameters) associated with the player. For example, injury risk assessment module 202 can access a player workload summary from workload summaries 241, can access a player injury history from injury histories 242, and can access a player match/game schedule from team schedules 243. Injury risk assessment module 202 can assess injury risk for the player based on the accessed player workload summary, the accessed player injury history, and the accessed player match/game schedule.

Player injury risk can be represented numerically in a range (e.g., 1-10), as a percentage, etc. Higher numbers or percentages can indicate increased injury risk and lower numbers or percentages can indicate decreased injury risk or vice versa. In one aspect, player injury risk is calculated as a number or percentage that is transformed to a categorical risk level, such as, for example, low risk, medium risk, or high risk. Injury risk assessment module 202 can assess injury risk per various different injury types (e.g., leg, arm, shoulder, back, etc.). For example, a player may be at lower risk for arm injury but at higher risk for (e.g., prone to) leg injury. As such, risk assessment module 202 can stratify/indicate player injury risk per injury type, for example, with a numerically ranged value, percentage, or categorical risk level indicated per the various different injury types.

In one aspect, risk assessment module 202 appends at least a portion of a player's injury history to the player's assessed injury risk. Alternately, risk assessment module 202 includes a link to the player's injury history in the players assessed injury risk.

Injury risk assessment module 202 can send an assessed player injury risk to risk prediction module 203. Risk prediction module 203 can receive the assessed player injury risk from injury risk assessment module 202. In general, risk prediction module 203 can predict future player injury risk for a player from an assessed player injury risk and in view of one or more of: purported upcoming player workload, a team training schedule, a team game/match schedule, or player risk factors.

As depicted, risk prediction module 203 further includes workload calculator 204, risk factor understanding module 206, and injury risk predictor 207. Workload calculator 204 can calculate a purported player workload for a player from a team schedule and training plan associated with the player's team. For example, workload calculator 204 can access a player training plan and/or a team training plan from training plans 244. Similarly, workload calculator 204 can access a team schedule from team schedules 243.

In one aspect, training plans are stored in training plans 244 per team and/or per player (e.g., by player identifier).

Thus, a purported player workload can include a training workload component and a match/game workload component. A purported player workload can include/indicate information similar to that stored in workload summaries 241 but is primarily forward looking instead of primarily historical. That is, a purported workload indicates upcoming workload that is to occur in the future (e.g., in the next day, in the next week, 2 days before the next match/game, 3 days after the next match/game, etc.), for example, based on upcoming training sessions and/or upcoming matches/games.

Risk factor understanding module 206 is configured to understand risk factors associated with and/or contributing to a player's injuries. Risk factor understanding module 206 can derive a player's injury risk factors from an assessed player injury risk and/or relevant portions of the player's injury history. Injury risk factors can include particular weather conditions, matches against particular teams, playing particular positions in a match/game, activities conduced in particular locations, etc.

Injury risk predictor 207 is configured to predict future player injury risk from one or more of: assessed player injury risk, purported (upcoming) player workload, a team training plan, a team match/game schedule, risk factors, etc. Injury risk predictor 207 can also access/consider information from other sources, such as, weather databases. For example, if rain predicted at an upcoming match or training session. Injury risk predictor 207 can represent predicted future player injury risk similar to assessed player injury risk, for example, using numerical ranges, percentages, etc. stratified/indicated across one or more various different injury types. Injury risk predictor 207 can send a predicted future player injury risk to risk threshold comparator 208.

Risk threshold comparator 208 can receive a predicted future player injury risk from injury risk predictor 207. In general, risk threshold module 208 is configured to compare predicted future player injury risk to player risk thresholds and determine if predicted future player injury risk is acceptable/appropriate or unacceptable/inappropriate. In one aspect, risk threshold comparator 208 compares a predicted future player injury risk to one or more corresponding risk thresholds. A risk threshold can be selected to flag predicted future player injury risk deemed to be unacceptable/inappropriate (e.g., to great).

Risk thresholds can be represented similarly to predicted future player injury risk and/or assessed player injury risk, for example, using numerical ranges, percentages, etc. and stratified/indicated across one or more various different injury types. As such, a predicted future player injury risk can be compared to risk threshold across the one or more various different injury types. Risk threshold comparator 208 can determine and flag any unacceptable/inappropriate player injury risk per injury type.

Risk threshold comparator 208 can notify team personnel and/or other modules whether player risk is acceptable/appropriate and/or unacceptable/inappropriate (e.g., per injury type). In one aspect, risk threshold comparator 207 sends an alert including predicted future player injury risk (or a portion thereof corresponding to a specific injury type) along with a corresponding risk threshold to template derivation module 209.

Template derivation module 209 receives the alert from risk threshold comparator 208. In general, template derivation module 209 is configured to derive a workload template defining a future workload that lowers predicted future player injury risk (e.g., down to an acceptable/appropriate risk). For example, template derivation module 209 can derive a workload template defining a future workload, that if implemented, would lower a player's future injury risk to satisfy specified risk thresholds for the player. Thus, template derivation module 209 can define a future workload that differs from a purported worked calculated by workload calculator 204. Workload differences can include varied (possibly reduced) repetitions, varied (possibly reduced) intensity, different activities, eliminated activities, new activities, resting, not participating in training, not participating in a match/game, etc.

Template derivation module 209 can notify team personnel or other systems, including sending a workload template along with a predicted future player injury risk. Team personnel can implement the defined future workload to reduce player injury risk while minimizing reduction in player workload.

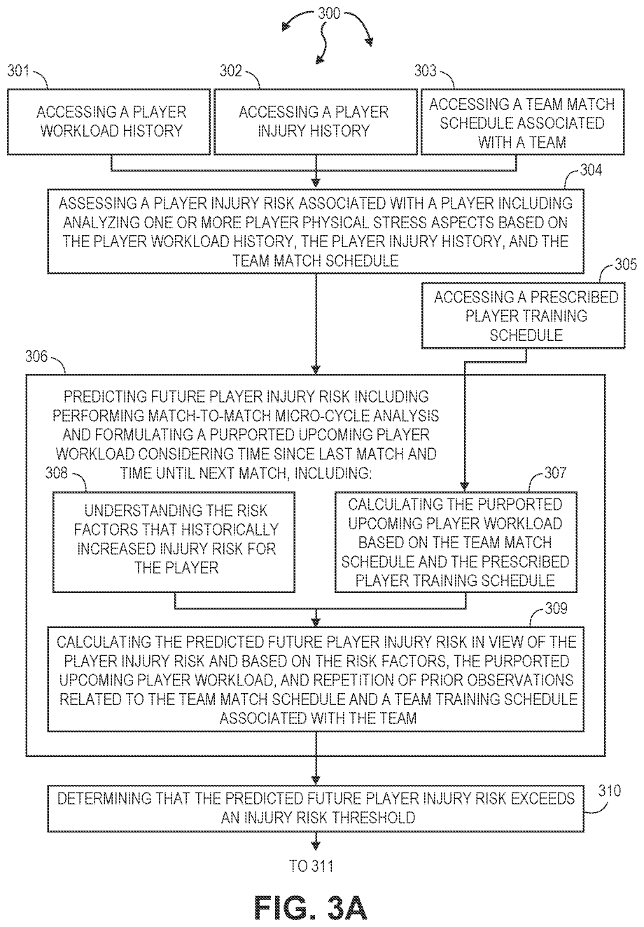

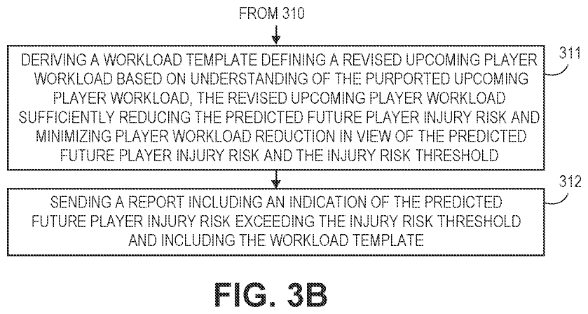

FIGS. 3A and 3B illustrates a flow chart of an example method 300 for predicting and mitigating athlete injury risk. Method 300 will be described with respect to the components and data in computer architecture 200.

Team personal or another system can input player identifier 211 to injury risk assessment module 202. Player identifier 211 can identify a particular player or athlete.

Method 300 includes accessing a player workload history (301). For example, injury risk assessment module 202 can use player identifier 211 to access corresponding player workload summary 212 from workload summaries 241. Method 300 includes accessing a player injury history (302). For example, injury risk assessment module 202 can use player identifier 211 to access corresponding player injury history 213 from injury histories 242. Method 200 includes accessing a team match schedule associated with a team (303). For example, injury risk assessment module 202 can use player identifier 211 to access schedule 214 from team schedules 243. Team schedule 243 can include prior and future games/matches associated with the particular player or athlete.

Method 300 includes assessing a player injury risk associated with a player including analyzing one or more player physical stress aspects based on the player workload history, the player injury history, and the team match schedule (304). For example, injury risk assessment module 202 can assess player injury risk 216 (for the particular player or athlete). Assessing player injury risk 216 includes analyzing player physical stress aspects (associated with the particular player or athlete) based on player workload summary 212, player injury history 213, and schedule 214.

Injury risk assessment module 202 may also access other inputs (not shown), such as, for example, player strength tests, player fitness tests, player wellbeing data, etc. Injury risk assessment module 202 can assess player injury risk including analyzing one or player physical stress aspects based on the other inputs (possibly in combination with a player workload history, a player injury history, and a team match schedule). For example, injury risk assessment module 202 can assess player injury risk 216 including analyzing player physical stress aspects based on one or more of: player strength tests, player fitness tests, player wellbeing data, etc. associated with player identifier 211. Depending at least in part on system configuration, different combinations of one or more described inputs can be analyzed and/or utilized when assessing player injury risk.

Assessment of player injury risk can trigger an alert to the player, a coach, a trainer, or other team personnel. The alert can be indicative of an assessed player injury risk. For example, injury risk assessment module 202 can send alert 228 (indicative of player injury risk 216) to team personnel 247 (e.g., a coach, a manager, a trainer, etc.). Team personnel is defined to include coaches, managers, trainers, etc. of athletes/players that participate in individual competitions, such as, track athletes, swimmers, tennis players, cyclists, runners, etc.

Injury risk assessment module 202 can also send player injury risk 216 to risk prediction module 203. Risk prediction module 203 can receive player injury risk 216 from injury risk assessment module 202. Risk prediction module 203 can use player identifier 211 to access schedule 214 from team schedules 243.

Method 300 includes accessing a prescribed player training schedule (305). For example, risk prediction module 203 can use player identifier 211 to access (player specific) training plan 217 from training plans 244. Risk prediction module 203 can also access team training plan 246 from training plans 244. There may be no team training plan when the particular player or athlete participates in individual sporting activities.

Method 300 includes predicting future player injury risk including performing match-to-match micro-cycle analysis and formulating a purported upcoming player workload considering time since last match (or event) and time until next match (or event) (306). For example, (and as part of further reporting associated with assessed player injury risk 216 and alert 228) risk prediction module 203 can determine predicted future player injury risk 221 (for the particular player or athlete) including performing match-to-match micro-cycle analysis on schedule 214 and formulating purported workload 218. Match-to-match micro-cycle analysis and workload formulation can consider time since the particular player's or athlete's last game (or event) and time until the particular player's or athlete's next game (or event).

Predicting future player injury risk includes calculating the purported upcoming player workload based on the team match schedule and the prescribed player training schedule (307). For example, workload calculator 204 can calculated purported workload 218 (for the particular player or athlete) based on schedule 214 and training plan 217. Predicting future player injury risk includes understanding the risk factors that historically increased injury risk for the player (308). For example, risk factor understanding module 206 can understand risk factors 219 (associated with the particular payer or athlete) based on player injury risk 216, possibly in combination with portions of injury history 213 (either included in player injury risk 216 or accessed from injury histories 242).

In one aspect, at least one unique category is included in the risk factors 219.

Predicting future player injury risk includes calculating the predicted future player injury risk in view of the player injury risk and based on the risk factors, the purported upcoming player workload, and repetition of prior observations related to the team match schedule and a team training schedule associated with the team (309). For example, injury risk calculator 207 can calculate predicted future player injury risk 221 (for the particular player or athlete) in view of player injury risk 216 and based on risk factors 219, purported workload 218, observations associated with schedule 214, and team training plan 246. A team training plan may not be considered, for example, the particular player or athlete competes individually in sporting events.

Injury risk calculator 207 can send predicted future player injury risk 221 to risk threshold comparator 208. Risk threshold comparator 208 can receive predicted future player injury risk 221 from injury risk calculator 207.

Method 300 includes determining that the predicted future player injury risk exceeds an injury risk threshold (310). For example, (and as part of further reporting associated with assessed player injury risk 216 and alert 228) risk threshold comparator 208 can compare predicted future player injury risk 221 and risk threshold 222. Risk threshold comparator 208 can determine that predicted future player injury risk 221 (for the particular player or athlete) exceeds risk threshold 222. In response, risk threshold comparator 208 can send notification 223, including predicted future player injury risk 221 and risk threshold 222 to template derivation module 209. Template derivation module 209 can receive alert 223 from risk threshold comparator 208.

Method 300 include deriving a workload template defining a revised upcoming player workload based on understanding of the purported upcoming player workload, the revised upcoming player workload sufficiently reducing the predicted future player injury risk and minimizing player workload reduction in view of the predicted future player injury risk and the injury risk threshold (311). For example, (and as part of further reporting associated with assessed player injury risk 216 and alert 228) template derivation module 209 can derive template 226. Template 226 can define a revised workload (for the particular player or athlete) that revises purported workload 218 based on an understanding of purported workload 218. The revised workload can sufficiently reduce predicted future player injury risk 221 and minimize workload reduction of the particular player or athlete in view of predicted future player injury risk 221 and the injury risk threshold 222.

In one aspect, template 226 is derived in view of unique category included in risk factors 219.

Method 300 includes sending a report including an indication of the predicted future player injury risk exceeding the injury risk threshold and including the workload template (312). For example, (and as part of further reporting associated with assessed player injury risk 216 and alert 228) template derivation module 209 can send report 224, including template 226 and predicted future player injury risk 221, to team personnel 247 (and/or directly to the particular player or athlete). Team personnel 247 (or the athlete/player) can utilize template 226 to reduce injury risk (e.g., below threshold 222) in a manner that minimizes workload reduction.

Accordingly, in response to an assessed player injury risk, an alert can be generated and sent to team personnel (and/or the player/athlete). A corresponding report can also be generated and sent to team personnel (and/or the player/athlete). The report can provide context on the alert (e.g., risk factors, injury types, etc.) and suggest an intervention plan (e.g., predicting future injury risk, creating a template, etc.).

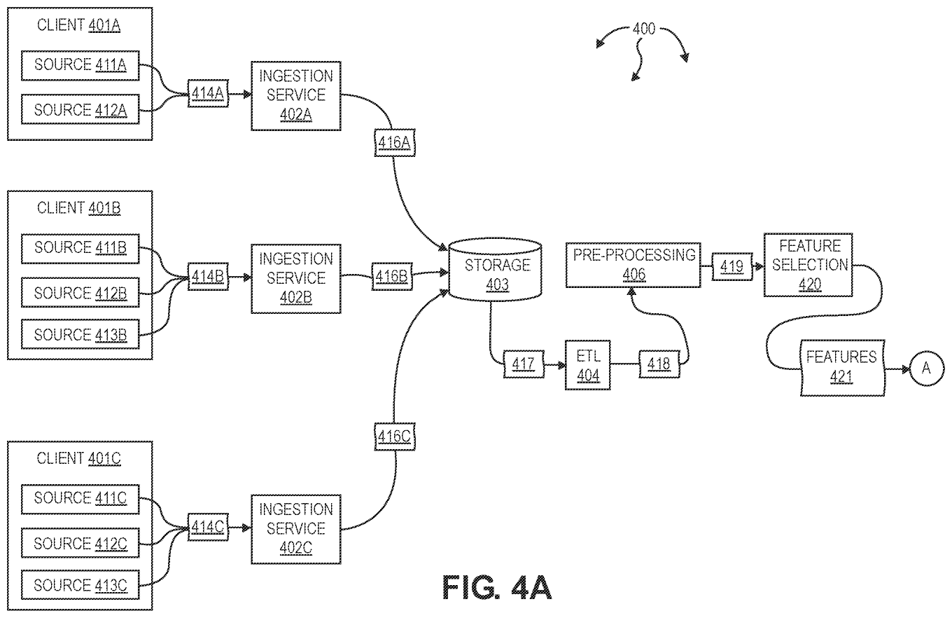

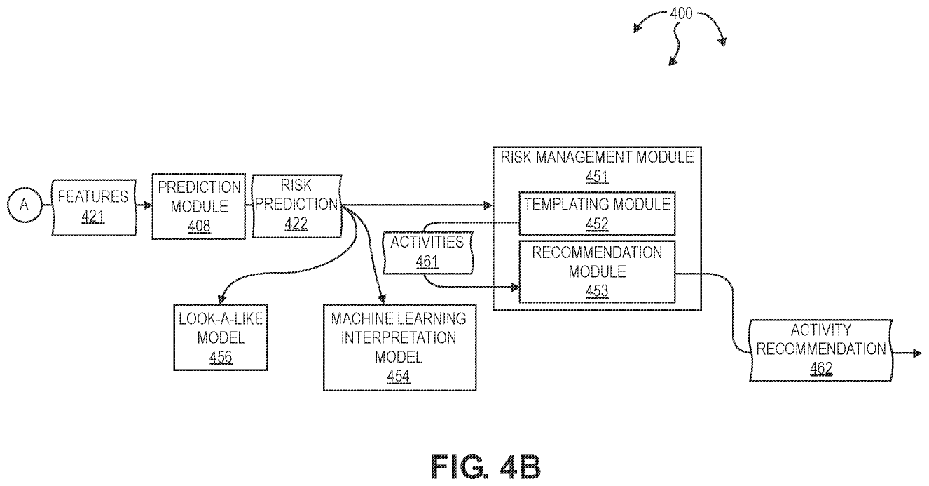

In some aspects, athlete data is collected from a plurality of clients and each client can include a plurality of data sources. FIGS. 4A and 4B illustrate an example computer architecture 400 for collecting data and making injury risk predictions. As depicted, computer architecture 400 includes clients 401A, 401B, and 401C, ingestion services 402A, 402B, and 402C, storage 403, ETL 404, pre-processing 406, feature selection 407, and prediction 408. Client 401A further includes data sources 411A and 412A. Client 401B further includes data sources 411B, 412B, and 413B. Client 401C further includes data sources 411C, 412C, and 413C.

In general, ingestion services can ingest athlete performance data (e.g., workload summaries, injury histories, team schedules, individual training plans, team training plans, etc.) from corresponding clients and store the ingested data in storage 403. Data from different clients and/or different data sources of a client can be stored in different formats, configurations, etc. Ingestion services may format (normalize) data from different clients into a common format compatible with other modules depicted in computer architectures 200, 400, 500, etc. Normalization during ingestion can facilitate more efficient and/or more effective processing of athlete performance data by other modules. For example, processor and memory resources can be conserved by normalizing data upon ingestion relative to multiple and varied data conversions later in athlete risk assessment/mitigation. In one aspect, artificial intelligence/machine learning is configured for a particular data format/configuration. Normalizing during ingestion frees up artificial intelligence/machine learning resources to assess and/or mitigate athlete injury risk.

Ingestion service 402A can ingest and normalize data 414A (e.g., athlete performance data associated with client 401A) from data sources 411A and 412A. Ingestion service 402A can combine, format, etc. data 414A into data 416A and store data 416A in storage 403 in a common format.

Ingestion service 402B can ingest data 414B (e.g., athlete performance data associated with client 401B) from data sources 411B, 412B, and 413B. Ingestion service 402B can combine, format, etc. data 414B into data 416B and store data 416B in storage 403 in the common format.

Ingestion service 402C can ingest data 414C (e.g., athlete performance data associated with client 401C) from data sources 411C, 412C, and 413C. Ingestion service 402C can combine, format, etc. data 414C into data 416C and store data 416C in storage 103 in the common format.

ETL 404 can extract data 417 (e.g., some merged combination of data from one or more of: 416A, 416B, and 416C) from storage 403, transform data 417 into data 418 (including further normalization), and load data 418 to pre-processing 406. Pre-processing 406 can pre-process data 418 into data 419 (including additional normalization and feature engineering) for feature selection. Feature selection 407 can select features 421 from data 419. Feature selection 407 can send features 421 to prediction module 408. Prediction module 408 (similar to risk prediction module 203) can make injury risk prediction 422 (e.g., for a particular athlete) from features 421. In one aspect, features 421 include one or more of: a previously assessed player injury risk, a player workload summary, a player injury history, a team schedule, an player training plan, a team training plan, a player purported workload, or player risk factors.

Turning to FIG. 4B, computer architecture 400 further includes risk management module 451, machine learning interpretation model 454, and look-a-like model 456. Risk management module 451 further includes templating module 452 and recommendation module 453. Prediction module 408 can send risk prediction 422 to risk management module 451, to machine learning interpretation model 454, and to look-a-like model 456.

Templating module 452 can use a templating based algorithm to identify activities 461 (e.g., activities that mitigate injury risk). Templating module 452 can send activities 461 to recommendation module 453. From among activities 461, recommendation module 453 can make activity recommendation 462. In one aspect, activity recommendation 462 is a recommendation for an activity that makes the slightest possible adjustment of an original activity (e.g., one of activities 461) that mitigates risk. For example, a training exercise can be modified enough to reduce athlete risk but yet still provide significant training benefit to the athlete.

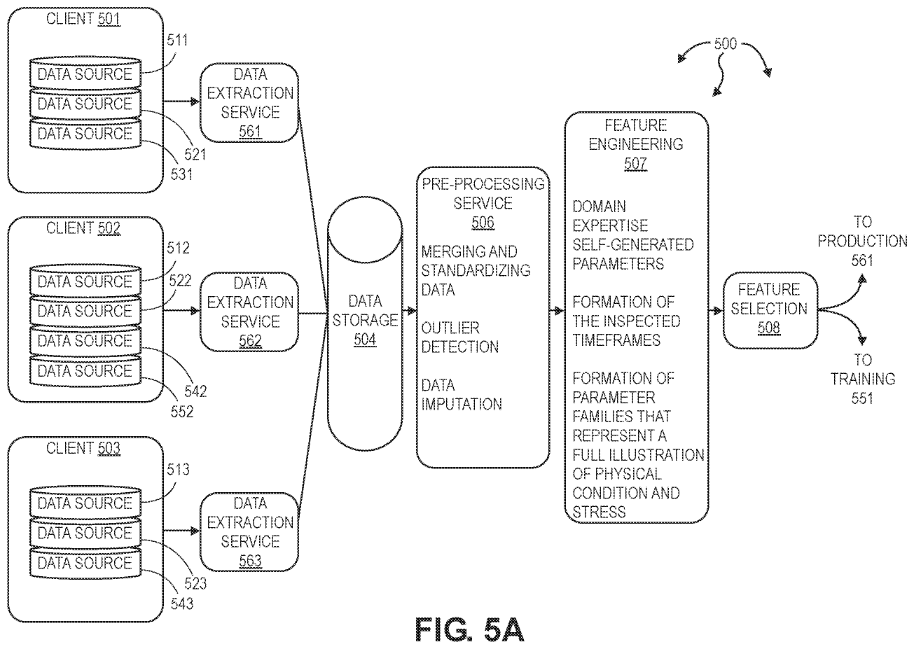

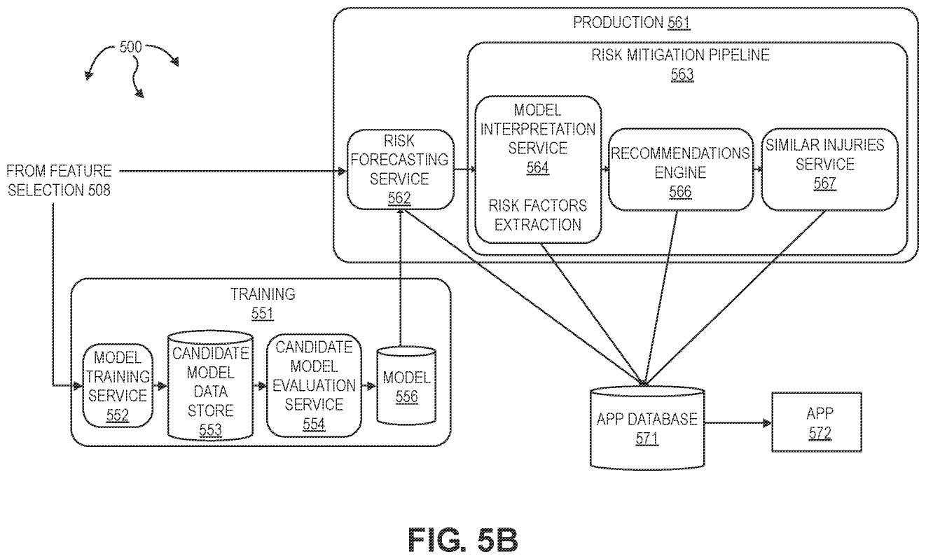

FIGS. 5A and 5B illustrate another example computer architecture 500 for collecting data and making injury risk predictions. Computer architecture 500 can operate similarly to computer architecture 400. As depicted, computer architecture 500 includes clients501, 502, and 502, data extraction services 561, 562, and 563, storage 504, pre-processing 506, feature selection 508, training 551, production 561, app database 571, and app 572.

Client 501 further includes data sources 511, 521, and 531. Client 502 further includes data sources 512, 522, 532, and 542. Client 503 further includes data sources 513, 523, and 543. Any of the such data source can be and/or include workload summaries, injury histories, team schedules, training plans, weather databases, etc.

Data extractions services 501, 502, 503, etc. can normalize data from different data sources into a common format for storage at data storage 504. Per-processing service 506 can perform further normalization in data in data storage 504 including merging and standardizing data, identifying outliers, and data imputation. Feature engineering 507 can utilize the further normalized data to self-generate domain expertise parameters, form inspected time frames, and form parameter families that represent athlete physical condition and stress. Feature selection 507 can select various features from the parameters and timeframes. Feature selection 507 can send the selected features to training 551 and production 561.

Training 551 can use the selected features for model training. Model training service 552 can execute models included in candidate module store 553 using the selected features. Candidate model evaluation service 554 can determine (e.g., best or appropriate) model 556 from among models in candidate model store 553. Risk forecasting service 562 can use model 556 when forecasting athlete injury risk.

Production 561 can be used to recommend athlete activities mitigating athlete injury risk. In one aspect, risk forecasting service 562 executes model 556 using the selected features to forecast athlete injury risk. Risk forecasting service 562 sends the forecasted risk to risk mitigation pipeline 563. Risk mitigation pipeline 563 can be applied for instances of data points over a risk threshold. Model interpretation service 564 can extract injury risk factors associated with the athlete. Recommendations engine 566 can recommend other actives to mitigate injury risk of the athlete while minimizing workload reduction. Similar injuries services 567 can consider similar injuries to other athletes based on forecasted risk and risk factors of the athlete.

In one aspect, a reference data point is created based on player workload history and the purported upcoming player workload. Player injury risk is then assessed by calculating a player risk score from the reference data point. A workload template is derived including retaining the reference data point as the workload template. Parameters compatible with the athlete injury risk factors for a specific injury risk alert are extracted. The reference data point is used to extract a minimum value for the parameter and a maximum value for the parameter.

Data from production 561 can be stored in app database 571 and sent to app 572.

In one aspect, modules from one or more of computer architectures 200, 400, or 500 interoperate to implement a training recommendation algorithm. The training recommendation algorithm can be implemented in accordance with the following (or other similar) flow:

1. Extract a player's training database under predefined restrictions (coach, team, relevant dates, etc.).

2. Extract the phase of the match-to-match micro-cycle that represents the next session.

3. Create reference data for the relevant phase of the micro-cycle based on prior sessions that took place in similar phases (or even the same exact phase).

4. Evaluate if any of the prior sessions from the reference data mitigate injury risk in a sufficient manner. For example, each session in the reference data can be evaluated as if it were the next session a player was to perform. By doing so, a risk generated score is associated with each session. Sessions associated with a risk score lower than a predefined risk threshold can be tagged as sessions that mitigate risk sufficiently. Sessions that sufficiently mitigate risk can be the selected workload templates from which a final recommendation may be formed.

5. If no session from the reference data is found to mitigate risk, the same (or similar) process of 4 can be performed using the player's entire training database as described in 1.

6. If a workload template consisting of multiple sessions was formed in 5, the workload template can be filtered so sessions that are (e.g., exponentially) more similar than others to the reference data remain (e.g., based on statistical similarity analysis). Filtering more similar sessions can be used to create a recommended workload template that both mitigates injury risk and is the slightest adjustment possible relative to the training sessions more likely planned based on match-to-match micro-cycle analysis.

7. If no sessions were found to mitigate risk in 4 and 5, the sessions that reduce the risk score to a minimal possible value can serve as the selected workload templates from which a final recommendation may be formed.

8. Each session in a risk mitigating workload template can include hundreds of parameters. As such, each session can be summarized for a human operator (e.g., a fitness coach or a sports scientist) to find it as an actionable recommendation. Accordingly, a final presented recommendation can include selected parameters and a range of suggested values for each parameter.

The selected parameters can be determined using machine learning interpretation modules (possibly in interoperation with other modules). The machine learning interpretation modules can extract a number of (e.g., up to five) parameters that contributed significantly (e.g., the most) to injury risk based on machine learning interpretation models (e.g., including, but not limited, to machine learning interpretation module 454), such as, a Shapley Additive Explanations (SHAP) method. Extracted parameters can serve as risk factors and can be selected for the final recommendation. However, in certain cases a user can select certain (and possibly different) parameters for a final recommendation due to practical reasons (e.g., having live data feeds for only a subset of parameters). A range associated with each parameter can be determined by analyzing each parameter's distribution within the risk mitigating workload template. Essentially, the range represents common values for each parameter specifically in the sessions selected for the workload template.

9. A user can be provided with context regarding each recommendation. The context can include a statement indicating if the recommendation represents an adjustment compared to (e.g., a minimized change from) the session that is most likely planned based on micro-cycle analysis. If changes are indicated, a list of reference dates from which the recommendation was built can be presented. Providing context, reference dates, etc., allows users to go back and understand how these sessions were structured and what needs to be done to perform similar sessions in practice.

Further, and in general, different modules from one or more of computer architectures 100, 200, 400, or 500 can interoperate to assess and/or mitigate athlete injury risk in a manner that minimizes athlete workload reduction. For example, modules of computer architecture 400 and/or computer architecture 500 can interoperate (potentially with modules of computer architecture 200) to implement method 300.

In the above disclosure, reference has been made to the accompanying drawings, which form a part hereof, and in which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Implementations of the systems, devices, and methods disclosed herein may comprise or utilize a special purpose or general-purpose computer including computer hardware, such as, for example, one or more processors and system memory, as discussed herein. Implementations within the scope of the present disclosure may also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations of the disclosure can comprise at least two distinctly different kinds of computer-readable media: computer storage media (devices) and transmission media.

Computer storage media (devices) includes RAM, ROM, EEPROM, CD-ROM, Solid State Drives (SSDs) (e.g., RAM-based or Flash-based), Shingled Magnetic Recording (SMR) devices, storage class memory (SCM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

In one aspect, one or more processors are configured to execute instructions (e.g., computer-readable instructions, computer-executable instructions, etc.) to perform any of a plurality of described operations. The one or more processors can access information from system memory and/or store information in system memory. The one or more processors can (e.g., automatically) transform information between different formats, such as, for example, between any of: player workload summaries, player injury histories, team schedules, player identifiers, assessed player injury risk, individual training plans, team training plans, risk factors, purported workloads, predicted future player injury risk, risk thresholds, alerts, templates, notifications, selected features, parameters, other athlete data, weather data, athlete risk predictions, look-a-like models, activities, machine learning interpretation modules, activity recommendations, parameters, models, athlete risk forecasts, application data, etc.

System memory can be coupled to the one or more processors and can store instructions (e.g., computer-readable instructions, computer-executable instructions, etc.) executed by the one or more processors. The system memory can also be configured to store any of a plurality of other types of data generated and/or transformed by the described components, such as, for example, player workload summaries, player injury histories, team schedules, player identifiers, assessed player injury risk, individual training plans, team training plans, risk factors, purported workloads, predicted future player injury risk, risk thresholds, alerts, templates, notifications, selected features, parameters, other athlete data, weather data, athlete risk predictions, look-a-like models, activities, machine learning interpretation modules, activity recommendations, parameters, models, athlete risk forecasts, application data, etc.

Implementations of the devices, systems, and methods disclosed herein may communicate over a computer network. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links, which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to computer storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that computer storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the description and claims to refer to particular system components. As one skilled in the art will appreciate, components may be referred to by different names. This document does not intend to distinguish between components that differ in name, but not function.

The described aspects can also be implemented in cloud computing environments. In this description and the following claims, “cloud computing” is defined as a model for enabling on-demand network access to a shared pool of configurable computing resources. For example, cloud computing can be employed in the marketplace to offer ubiquitous and convenient on-demand access to the shared pool of configurable computing resources (e.g., compute resources, networking resources, and storage resources). The shared pool of configurable computing resources can be provisioned via virtualization and released with low effort or service provider interaction, and then scaled accordingly.

A cloud computing model can be composed of various characteristics such as, for example, on-demand self-service, broad network access, resource pooling, rapid elasticity, measured service, and so forth. A cloud computing model can also expose various service models, such as, for example, Software as a Service (“SaaS”), Platform as a Service (“PaaS”), and Infrastructure as a Service (“IaaS”). A cloud computing model can also be deployed using different deployment models such as on premise, private cloud, community cloud, public cloud, hybrid cloud, and so forth. In this description and in the following claims, a “cloud computing environment” is an environment in which cloud computing is employed.

Hybrid cloud deployment models combine portions of other different deployment models, such as, for example, a combination of on premise and public, a combination of private and public, a combination of two different public cloud deployment models, etc. Thus, resources utilized in a hybrid cloud can span different locations, including on premise, private clouds, (e.g., multiple different) public clouds, etc.

At least some embodiments of the disclosure have been directed to computer program products comprising such logic (e.g., in the form of software) stored on any computer useable medium. Such software, when executed in one or more data processing devices, causes a device to operate as described herein.

While various embodiments of the present disclosure have been described above, it should be understood that they have been presented by way of example only, and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the following claims and their equivalents. The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations may be used in any combination desired to form additional hybrid implementations of the disclosure.

1. A method comprising:
accessing a player workload history;
accessing a player injury history;
accessing a team match schedule associated with a team;
assessing a player injury risk associated with a player including analyzing one or more player physical stress aspects based on the player workload history, the player injury history, and the team match schedule;
accessing a prescribed player training schedule;
predicting future player injury risk including performing match-to-match micro-cycle analysis and formulating a purported upcoming player workload considering time since last match and time until next match, including:
calculating the purported upcoming player workload based on the team match schedule and the prescribed player training schedule;
understanding the risk factors that historically increased injury risk for the player; and
calculating the predicted future player injury risk in view of the player injury risk and based on the risk factors, the purported upcoming player workload, and repetition of prior observations related to the team match schedule and a team training schedule associated with the team;
determining that the predicted future player injury risk exceeds an injury risk threshold;
deriving a workload template defining a revised upcoming player workload based on understanding of the purported upcoming player workload, the revised upcoming player workload sufficiently reducing the predicted future player injury risk and minimizing player workload reduction in view of the predicted future player injury risk and the injury risk threshold; and
sending a report including an indication of the predicted future player injury risk exceeding the injury risk threshold and including the workload template.

2. The method of claim 1, wherein performing match-to-match micro-cycle analysis comprises determining a match-to-match micro-cycle phase is one day before a match.

3. The method of claim 1, wherein deriving a workload template comprises identifying a training session.

4. The method of claim 1, wherein deriving a workload template comprises:
identifying at least one unique category included in the risk factors; and
deriving workload template in view of the at least one unique category.

5. The method of claim 4, wherein assessing a player injury risk comprises:
creating a reference data point based on the player workload history and the purported upcoming player workload; and
calculating a player risk score from the reference data point.

6. The method of claim 5, wherein deriving the workload template comprises retaining the reference data point as the workload template; and
further comprising:
extracting parameters compatible with the risk factors for a specific injury risk alert; and
using the reference data point to extract a minimum value for the parameter and a maximum value for the parameter.

7. The method of claim 1, further comprising accessing one or more of: player strength tests, player fitness tests, or player wellbeing data; and
wherein accessing a player injury risk associated with the player comprises assessing the player injury risk including analyzing the one or more player physical stress aspects based on the one or more of: player strength tests, player fitness tests, or player wellbeing data.

8. The method of claim 1, further comprising sending an alert indicative of the assessed a player injury risk to team personnel associated with the player; and
wherein sending a report comprises sending the report to the team personnel.

9. A computer system comprising:
a processor;
system memory coupled to the processor and storing instructions configured to cause the processor to:
access a player workload history;
access a player injury history;
access a team match schedule associated with a team;
assess a player injury risk associated with a player including analyzing one or more player physical stress aspects based on the player workload history, the player injury history, and the team match schedule;
access a prescribed player training schedule;
predict future player injury risk including performing match-to-match micro-cycle analysis and formulating a purported upcoming player workload considering time since last match and time until next match, including:
calculate the purported upcoming player workload based on the team match schedule and the prescribed player training schedule;
understand the risk factors that historically increased injury risk for the player; and
calculate the predicted future player injury risk in view of the player injury risk and based on the risk factors, the purported upcoming player workload, and repetition of prior observations related to the team match schedule and a team training schedule associated with the team;
determine that the predicted future player injury risk exceeds an injury risk threshold;
derive a workload template defining a revised upcoming player workload based on understanding of the purported upcoming player workload, the revised upcoming player workload sufficiently reducing the predicted future player injury risk and minimizing player workload reduction in view of the predicted future player injury risk and the injury risk threshold; and
send a report including an indication of the predicted future player injury risk exceeding the injury risk threshold and including the workload template.

10. The system of claim 9, wherein instructions configured to perform match-to-match micro-cycle analysis comprise instructions configured to determine a match-to-match micro-cycle phase is one day before a match.

11. The system of claim 9, wherein instructions configured to derive a workload template comprise instructions configured to identify a training session.

12. The system of claim 9, wherein instructions configured to derive a workload template comprise instructions configured to:
identify at least one unique category included in the risk factors; and
derive workload template in view of the at least one unique category.

13. The system of claim 12, wherein instructions configured to assess a player injury risk comprise instructions configured to:
create a reference data point based on the player workload history and the purported upcoming player workload; and
calculate a player risk score from the reference data point.

14. The system of claim 13, wherein instructions configured to derive the workload template comprise instructions configured to retain the reference data point as the workload template; and
further comprising instructions configured to:
extract parameters compatible with the risk factors for a specific injury risk alert; and
use the reference data point to extract a minimum value for the parameter and a maximum value for the parameter.

15. The system of claim 9, further comprising instructions configured to access one or more of: player strength tests, player fitness tests, or player wellbeing data; and
wherein instructions configured to access a player injury risk associated with the player comprise instructions configured to assess the player injury risk including analyzing the one or more player physical stress aspects based on the one or more of: player strength tests, player fitness tests, or player wellbeing data.

16. The method of claim 9, further comprising instructions configured to send an alert indicative of the assessed a player injury risk to team personnel associated with the player; and
wherein instructions configured to send a report comprise instructions configured to send the report to the team personnel.
